(19) United States
(12) Patent Application Publication
(10) Pub. No.: US 2022/0347433 A1
(43) Pub. Date: Nov. 3, 2022

(54) Magnetically Trackable Stylets and Methods Thereof

(71) Applicant: Bard Access Systems, Inc., Salt Lake City, UT (US)

(72) Inventors: Michael Davis, West Jordan, UT (US); Breanna E. Lackey, West Valley City, UT (US); Edward D. Bell, Kearns, UT (US); Robin Scott Urry, Layton, UT (US); Daniel B. Blanchard, Bountiful, UT (US); Shawn Ray Isaacson, Layton, UT (US); Bradley J. VanderStek, West Bountiful, UT (US); Taylor Matthew Eley, Salt Lake City, UT (US); Austin Russon, South Jordan, UT (US); Huy Ngoc Tran, Riverton, UT (US)

(21) Appl. No.: 17/731,169

(22) Filed: Apr. 27, 2022

Related U.S. Application Data
(60) Provisional application No. 63/181,071, filed on Apr. 28, 2021.
(60) Provisional application No. 63/181,060, filed on Apr. 28, 2021.

(51) Int. Cl. A61M 25/01

(52) U.S. Cl. CPC A61M 25/0127; A61M 25/0102; A61B 5/062

(57) ABSTRACT

Disclosed herein are magnetically trackable stylets and methods thereof. A magnetically trackable stylet can include a stylet body including a core wire, a magnetic assembly, and an outer construction over the core wire and the magnetic assembly. The magnetic assembly can include one or more magnetic field-producing elements disposed alongside the core wire in a magnetically trackable distal portion of the stylet body. The outer construction, which can be an overmolded layer, a reflowed layer, a potting layer, or a shrink-wrapped layer, can be around the core wire and the magnetic assembly. The stylet body can be configured to be disposed in a lumen of a medical device such as a catheter for magnetically tracking a tip of the medical device in vivo without breakage of the stylet body due to bending-related fatigue. A method of such a magnetically trackable stylet can include a method of using the stylet.

### PRIORITY

This application claims the benefit of priority to U.S. Provisional Patent Application No. 63/181,060, filed Apr. 28, 2021, and U.S. Provisional Patent Application No. 63/181,071, filed Apr. 28, 2021, each of which is incorporated by reference in its entirety into this application.

### BACKGROUND

The Sherlock 3CG™ Tip Confirmation System and the Sherlock 3CG+TCS (collectively, “TCS”) is indicated for guidance and positioning of peripherally inserted central catheters (“PICCs”). The TCS provides such guidance and positioning with real-time location information for PICC tips using passive magnet tracking and cardiac electrical activity for each patient. As such, the TCS is an advantageous alternative to chest X-rays and fluoroscopy for placing PICCs in adult patients, particularly when relying on patients' electrocardiography (“ECG”) signals. Because PICCs can be positioned up to 5 times faster with both fewer malpositions and reduced X-ray exposure using the TCS, the TCS continues to be important for guiding and positioning PICCs.

Disclosed herein are magnetically trackable stylets and methods thereof for the TCS or other such systems for medical device placement that utilize at least magnetic tracking for the medical device placement.

### SUMMARY

Disclosed herein is a magnetically trackable stylet including, in some embodiments, a stylet body including a core wire, a flexible magnetic assembly, and a casing. The magnetic assembly includes one or more magnetic field-producing elements disposed alongside the core wire in a magnetically trackable distal portion of the stylet body. The casing is around the core wire and the magnetic assembly. The stylet body is configured to be disposed in a lumen of a catheter for magnetically tracking a catheter tip in vivo without breakage of the stylet body due to bending-related fatigue.

In some embodiments, the core wire is tapered in the distal portion of the stylet body alongside the one-or-more magnetic field-producing elements.

In some embodiments, the stylet further includes a sealed stylet tip. The stylet tip includes a seal sealing the one-or-more magnetic field-producing elements in the distal portion of the stylet body.

In some embodiments, the one-or-more magnetic field-producing elements include one or more polymer-bonded magnets. The one-or-more polymer-bonded magnets are configured to bend and, thereby, allow the stylet to bend in accordance with an anatomy of a patient without breaking.

In some embodiments, the one-or-more polymer-bonded magnets include a single cylindrical polymer-bonded magnet.

In some embodiments, the one-or-more polymer-bonded magnets include a plurality of cylindrical polymer-bonded magnets.

In some embodiments, the one-or-more magnetic field-producing elements include one-or-more sintered magnets.

In some embodiments, the one-or-more sintered magnets include a plurality of cylindrical sintered magnets having radiused ends. The radiused ends of the cylindrical magnets configured to allow the cylindrical magnets to bend into each other and, thereby, allow the stylet to bend in accordance with an anatomy of a patient without breaking.

In some embodiments, the one-or-more sintered magnets include a plurality of cylindrical sintered magnets alternating with a plurality of spherical sintered magnets. The alternating cylindrical and spherical magnets form articulable joints therebetween. The joints are configured to allow the cylindrical magnets to bend into each other and, thereby, allow the stylet to bend in accordance with an anatomy of a patient without breaking.

In some embodiments, the one-or-more sintered magnets include a plurality of cylindrical sintered magnets alternating with a plurality of non-metallic spheres. The alternating cylindrical magnets and non-metallic spheres form articulable joints therebetween. The joints are configured to allow the cylindrical magnets to bend into each other and, thereby, allow the stylet to bend in accordance with an anatomy of a patient without breaking.

In some embodiments, the one-or-more sintered magnets include a plurality of cylindrical sintered magnets alternating with a plurality of septa sectioning a groove of a magnet holder in which the sintered magnets are disposed. The septa form articulable joints between the cylindrical magnets. The joints are configured to allow the cylindrical magnets to bend into each other and, thereby, allow the stylet to bend in accordance with an anatomy of a patient without breaking.

In some embodiments, the one-or-more sintered magnets include a plurality of cylindrical sintered magnets disposed in a plurality of magnet holders forming articulable joints therebetween. The joints are configured to allow the cylindrical magnets to bend into each other and, thereby, allow the stylet to bend in accordance with an anatomy of a patient without breaking.

In some embodiments, each magnet holder of the magnet holders includes a ball end and a socket end. The joints between the magnet holders are ball-and-socket joints.

In some embodiments, the magnet holders include ball-ended magnet holders having a pair of ball ends and socket-ended magnet holders having a pair of socket ends. The joints between the magnet holders are ball-and-socket joints.

In some embodiments, the magnet holders are links and the joints are interlinks between the links chained together.

In some embodiments, the one-or-more magnetic field-producing elements include one or more magnetic wires twisted or braided with the core wire. The one-or-more magnetic wires are configured to bend and, thereby, allow the stylet to bend in accordance with an anatomy of a patient without breaking.

Disclosed herein is a magnetically trackable stylet including, in some embodiments, a stylet body including a flexible magnetic assembly and a casing. The magnetic assembly includes one or more magnetic wires in a magnetically trackable distal portion of the stylet body. The casing is around the magnetic assembly. The stylet body is configured to be disposed in a lumen of a catheter for magnetically tracking a catheter tip in vivo without breakage of the stylet body due to bending-related fatigue.

In some embodiments, the one-or-more magnetic wires include a single magnetic wire.

In some embodiments, the stylet further includes a core wire. The magnetic wire is twisted with the core wire in the distal portion of the stylet body.

In some embodiments, the stylet further includes a core wire. The magnetic wire is helically wrapped around the core wire in the distal portion of the stylet body.

In some embodiments, the one-or-more magnetic wires include a plurality of magnetic wires.

In some embodiments, the stylet further includes a core wire. The magnetic wires are twisted or braided with the core wire in the distal portion of the stylet body.

In some embodiments, the stylet further includes a core wire. The magnetic wires are twisted or braided around the core wire in the distal portion of the stylet body.

In some embodiments, the stylet further includes a sealed stylet tip. The stylet tip includes a seal sealing the magnetic wires in the distal portion of the stylet body.

Disclosed herein is a magnetically trackable stylet including, in some embodiments, a stylet body including a core wire, a magnetic assembly, and an outer construction over the core wire and the magnetic assembly. The magnetic assembly includes one or more magnetic field-producing elements disposed alongside the core wire in a magnetically trackable distal portion of the stylet body. The outer construction is selected from the group consisting of an overmolded layer, a reflowed layer, a potting layer, and a shrink-wrapped layer. The stylet body is configured to be disposed in a lumen of a catheter for magnetically tracking a catheter tip in vivo without breakage of the stylet body due to bending-related fatigue.

In some embodiments, the outer construction is a single-layered outer construction.

In some embodiments, the outer construction includes the overmolded layer. The overmolded layer is molded around the core wire and the magnetic assembly.

In some embodiments, one or more gaps between any two or more magnetic field-producing elements include polymeric material of the overmolded layer therebetween. The one-or-more gaps with the polymeric material form one or more articulable joints in the magnetic assembly.

In some embodiments, the outer construction includes the reflowed layer. The reflowed layer is reflowed around the core wire and the magnetic assembly.

In some embodiments, one or more gaps between any two or more magnetic field-producing elements include polymeric material of the reflowed layer therebetween. The one-or-more gaps with the polymeric material form one or more articulable joints in the magnetic assembly.

In some embodiments, the outer construction includes the potting layer. The potting layer is potted around the core wire and the magnetic assembly.

In some embodiments, one or more gaps between any two or more magnetic field-producing elements include potting material of the potting layer therebetween. The one-or-more gaps with the potting material form one or more articulable joints in the magnetic assembly.

In some embodiments, the outer construction includes the shrink-wrapped layer. The shrink-wrapped layer is shrunk around the core wire and the magnetic assembly.

In some embodiments, one or more gaps between any two or more magnetic field-producing elements form one or more articulable joints in the magnetic assembly.

In some embodiments, the outer construction is a multi-layered outer construction.

In some embodiments, the outer construction includes the overmolded layer and one or more other layers over the overmolded layer. The overmolded layer is molded around the core wire and the magnetic assembly.

In some embodiments, one or more gaps between any two or more magnetic field-producing elements include polymeric material of the overmolded layer therebetween. The one-or-more gaps with the polymeric material form one or more articulable joints in the magnetic assembly.

In some embodiments, the one-or-more other layers include a casing disposed over the overmolded layer.

In some embodiments, the outer construction includes the reflowed layer and one or more other layers over the reflowed layer. The reflowed layer is reflowed around the core wire and the magnetic assembly.

In some embodiments, one or more gaps between any two or more magnetic field-producing elements include polymeric material of the reflowed layer therebetween. The one-or-more gaps with the polymeric material form one or more articulable joints in the magnetic assembly.

In some embodiments, the one-or-more other layers include a braided layer over the reflowed layer and an outer casing over the braided layer. The reflowed layer is reflowed into the braided layer.

In some embodiments, the outer construction includes the potting layer and one or more other layers over the potting layer. The potting layer is potted around the core wire and the magnetic assembly.

In some embodiments, one or more gaps between any two or more magnetic field-producing elements include potting material of the potting layer therebetween. The one-or-more gaps with the potting material form one or more articulable joints in the magnetic assembly.

In some embodiments, the one-or-more other layers include a casing disposed over the potting layer.

In some embodiments, the outer construction includes the shrink-wrapped layer and one or more other layers over the shrink-wrapped layer. The shrink-wrapped layer is shrunk around the core wire and the magnetic assembly.

In some embodiments, one or more gaps between any two or more magnetic field-producing elements form one or more articulable joints in the magnetic assembly.

In some embodiments, the one-or-more other layers include a casing disposed over the shrink-wrapped layer.

In some embodiments, the core wire is tapered in the distal portion of the stylet body alongside the one-or-more magnetic field-producing elements.

In some embodiments, the stylet further includes a sealed stylet tip. The stylet tip includes a seal sealing the one-or-more magnetic field-producing elements in the distal portion of the stylet body.

These and other features of the concepts provided herein will become more apparent to those of skill in the art in view of the accompanying drawings and following description, which describe particular embodiments of such concepts in greater detail.

### DRAWINGS

FIG. 1 illustrates a block diagram depicting various elements of a first integrated system for placement of a medical device such as a catheter within a patient in accordance with some embodiments.

FIG. 2 illustrates a simplified view of the patient and the catheter being placed within the patient using the first integrated system in accordance with some embodiments.

FIG. 3 illustrates an ultrasound image on a display of the first integrated system in accordance with some embodiments.

FIG. 4 illustrates a perspective view of a first stylet of the first integrated system in accordance with some embodiments.

FIG. 5A illustrates a graphical representation on the display of the first integrated system while placing the catheter but before a signal is received by a tip location sensor in accordance with some embodiments.

FIG. 5B illustrates a graphical representation on the display of the first integrated system with a signal received at a periphery of the tip location sensor in accordance with some embodiments.

FIG. 5C illustrates a graphical representation on the display of the first integrated system with a signal received beneath the tip location sensor in accordance with some embodiments.

FIG. 6 illustrates a block diagram depicting various elements of a second integrated system for placement of a medical device such as a catheter within a patient in accordance with some embodiments.

FIG. 7 illustrates a simplified view of the patient and the catheter being placed within the patient using the second integrated system in accordance with some embodiments.

FIG. 8 illustrates a perspective view of a second stylet of the second integrated system in accordance with some embodiments.

FIG. 9 illustrates a simplified view of an ECG trace of a patient in accordance with some embodiments.

FIG. 10 illustrates a graphical representation on a display of the second integrated system with a signal received beneath a tip location sensor in accordance with some embodiments.

FIG. 11 illustrates a stylet body of the first or second stylet in accordance with some embodiments.

FIG. 12 illustrates a detailed cross-sectional view of a distal portion of the first or second stylet in accordance with some embodiments.

FIG. 13 illustrates a detailed cross-sectional view of the distal portion of the first or second stylet in accordance with some embodiments.

FIG. 14 illustrates a detailed cross-sectional view of the distal portion of the first or second stylet in accordance with some embodiments.

FIG. 15A illustrates a detailed cross-sectional view of the distal portion of the first or second stylet in accordance with some embodiments.

FIG. 15B illustrates a detailed cross-sectional view of the distal portion of the stylet of FIG. 15A rotated 90° along an axis of stylet in accordance with some embodiments.

FIG. 16 illustrates a detailed cross-sectional view of the distal portion of the first or second stylet in accordance with some embodiments.

FIG. 17 illustrates a detailed cross-sectional view of the distal portion of the first or second stylet in accordance with some embodiments.

FIG. 18 illustrates a detailed cutaway view of the distal portion of the first or second stylet in accordance with some embodiments.

FIG. 19 illustrates a detailed cutaway view of the distal portion of the first or second stylet in accordance with some embodiments.

FIG. 20A illustrates a detailed cross-sectional view of a distal portion of the first or second stylet including a first outer construction in accordance with some embodiments.

FIG. 20B illustrates a detailed cross-sectional view of a distal portion of the first or second stylet including a second outer construction in accordance with some embodiments.

FIG. 20C illustrates a detailed cross-sectional view of a distal portion of the first or second stylet including a third outer construction in accordance with some embodiments.

FIG. 21A illustrates a detailed cross-sectional view of a distal portion of the first or second stylet including a fourth outer construction in accordance with some embodiments.

FIG. 21B illustrates a detailed cross-sectional view of a distal portion of the first or second stylet including a fifth outer construction in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, the TCS continues to be an advantageous alternative to chest X-rays and fluoroscopy for placing PICCs in adult patients, particularly when relying on patients' electrocardiography (“ECG”) signals. Disclosed herein are magnetically trackable stylets and methods thereof for the TCS or other such systems for medical device placement that utilize at least magnetic tracking for the medical device placement.

The drawings depict features of various embodiments of an integrated system for placing medical devices such as catheters within vasculatures of patients. In some embodiments, the integrated system employs at least two modes for improving accuracy of medical device placement: 1) an ultrasound (“US”) mode for introducing a medical device (e.g., the catheter 72) into a patient's vasculature under US visualization; and 2) a tip location sensor (“TLS”) mode for TLS or magnetic tracking of a tip of the medical device (e.g., the distal tip 76A of the catheter 72) during its advancement through tortuous vasculature, which, in turn, allows for detection and correction of any malposition of the medical device during such advancement. The US visualization and TLS tracking of the integrated system are, in some embodiments, integrated into a single integrated device for use by a clinician placing the medical device. Integration of the features of these two modes into the integrated device simplifies placement of medical devices and results in relatively faster placement of the medical devices. For instance, the integrated system enables US guidance and TLS tracking to be viewed from a single display of the integrated system. Also, controls located on a US probe of the integrated device, which probe is maintained within the sterile field of the patient during placement of the medical device, can be used to control functionality of the integrated system, thus precluding the need for a clinician to reach out of a sterile field in order to control the integrated system.

In some embodiments, a third mode, namely an ECG mode, is included in the integrated system to enable ECG confirmation of the tip of the medical device in a desired location with respect to a node of the patient's heart from which ECG signals originate.

Combination of features of the three modes set forth above enables the integrated system to facilitate placement of the medical device within the patient's vasculature with a relatively high level of accuracy. Moreover, because of the ECG confirmation for the tip of the medical device, correct tip placement can be confirmed without the need for a confirmatory X-ray. This, in turn, reduces the patient's exposure to potentially harmful X-rays, the cost and time involved in transporting the patient to and from the X-ray department, costly and inconvenient repositioning procedures, etc.

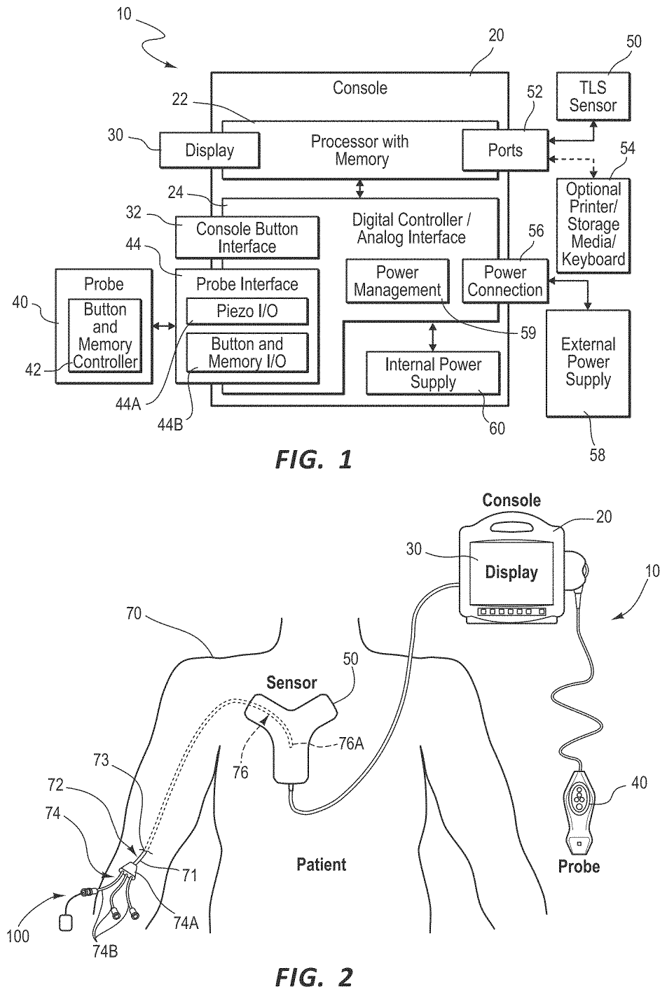

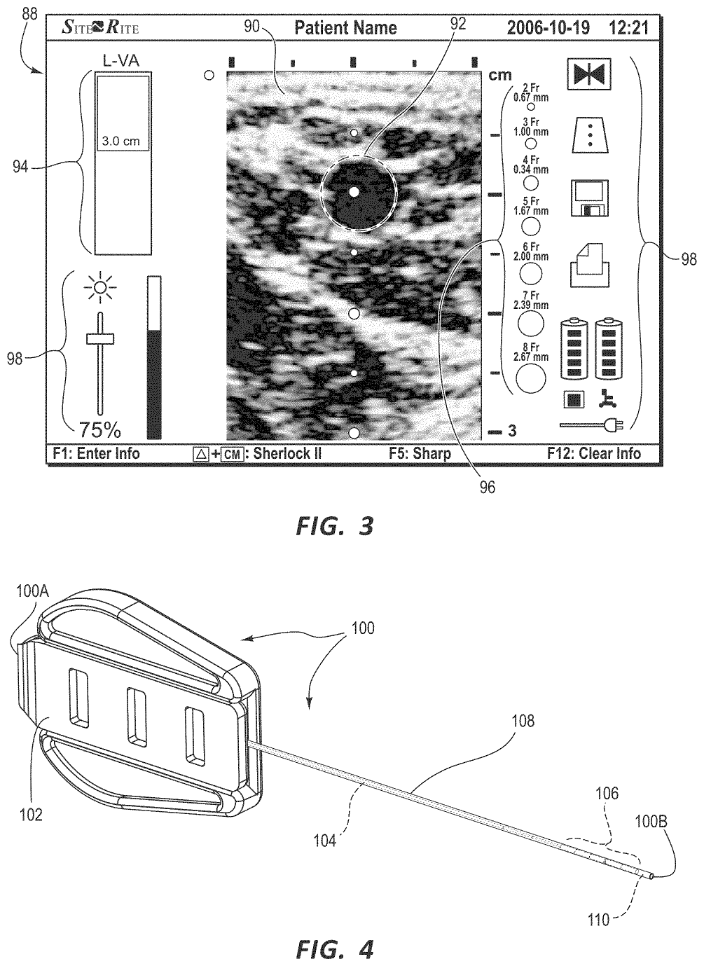

Reference is first made to FIGS. 1 and 2, which depict various components of an integrated system 10 for placing medical devices such as the catheter 72 thereof. As shown, the integrated system 10 generally includes a console 20, a display 30, a probe 40, and a TLS 50, each of which is described in further detail below.

FIG. 2 shows the general relation of the various components to a patient 70 during a procedure to place a catheter 72 (e.g., a PICC, a central venous catheter [“CVC”], or other suitable catheter) into the patient's vasculature through an insertion site 73 in the patient's skin. FIG. 2 shows that the catheter 72 generally includes a proximal portion 74 that remains exterior to the patient 70 and a distal portion 76 that resides within the patient's vasculature after placement is complete. The integrated system 10 is employed to ultimately position a distal tip 76A of the catheter 72 in a desired position within the patient's vasculature. In some embodiments, the desired position for the distal tip 76A of the catheter 72 is proximate the patient's heart, such as in the lower one-third of the Superior Vena Cava (“SVC”). However, the integrated system 10 can be employed to place the distal tip 76A of the catheter 72 in other locations as needed. The proximal portion 74 of the catheter 72 further includes a hub 74A that provides fluid communication between one or more lumens of a catheter tube 71 and one or more extension legs 74B proximally extending from the hub 74A.

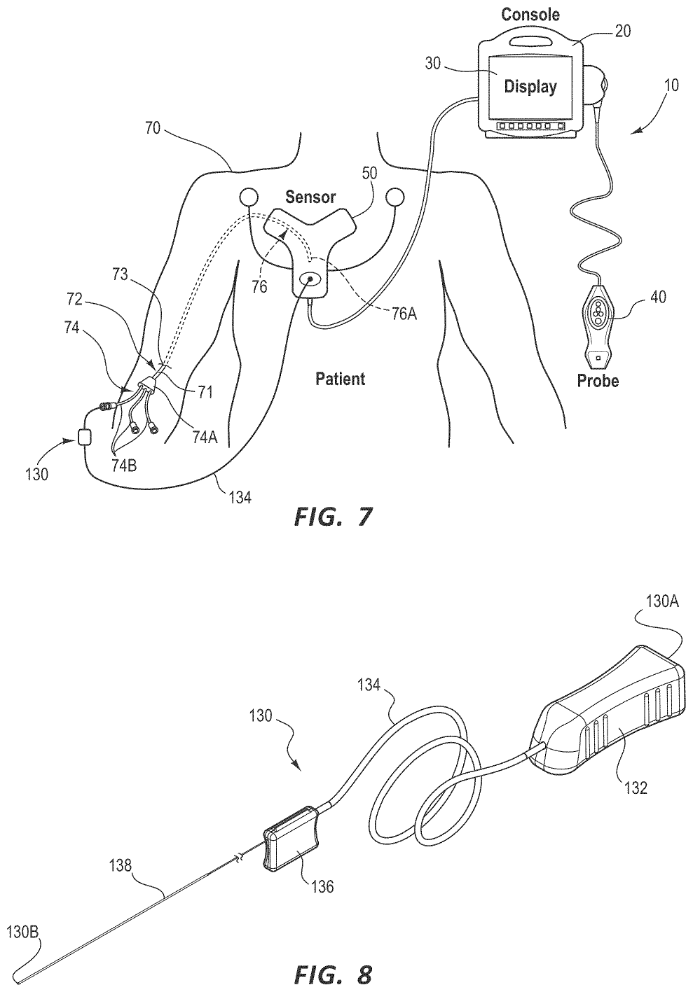

An example implementation of the console 20 is shown in FIG. 5C, though it is appreciated that the console 20 can take any of a variety of forms. As shown in FIG. 1, a processor 22 including non-volatile memory such as electrically erasable, programmable read-only memory (“EEPROM”) can be included in the console 20 for controlling system function during operation of the integrated system 10, thus acting as a control processor. A digital controller/analog interface 24 is also included with the console 20. The digital controller/analog interface 24 is in communication with both the processor 22 and other system components to govern interfacing between the probe 40, the TLS 50, and other system components.

The integrated system 10 further includes ports 52 for connection with the TLS 50 and optional components 54 including a printer, storage media, keyboard, etc. The ports 52 in some embodiments are USB ports; however, other port types or a combination of port types can be used for this and the other interfaces connections described herein. A power connection 56 is included with the console 20 to enable operable connection to an external power supply 58. An internal power supply 60 (e.g., a battery) can also be employed, either with or exclusive of the external power supply 58. Power management circuitry 59 is included with the digital controller/analog interface 24 of the console 20 to regulate power use and distribution.

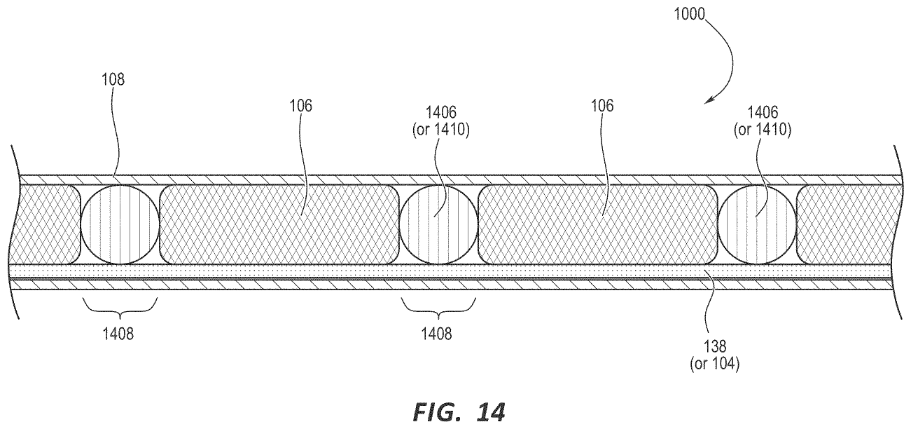

The display 30 can be integrated into the console 20. The display 30 is used to display information to a clinician during a placement procedure. In some embodiments, the display 30 can be separate from the console 20. The content depicted by the display 30 changes according to the mode(s) (e.g., the US mode, the TLS mode, the ECG mode, or a combination thereof) in use on the integrated system 10. In some embodiments, a console button interface 32 (see FIGS. 1 and 5C) and buttons included on the probe 40 can be used to immediately call up a desired mode to the display 30 by the clinician to assist in the placement procedure. In some embodiments, information from multiple modes such as the TLS and ECG modes can be displayed simultaneously. (See FIG. 10.) Thus, the display 30 of the console 20 can be employed for US visualization for accessing a patient's vasculature, TLS tracking during advancement of the catheter 72 through the vasculature, and ECG confirmation for confirming placement of the distal tip 76A of the catheter 72 with respect to a node of the patient's heart. In some embodiments, the display 30 is an LCD device.

The probe 40 shown in FIG. 2 is employed in connection with the first mode (i.e., the US mode) for US visualization of a vessel, such as a vein, in preparation for insertion of the catheter 72 into the vasculature. Such visualization gives real time assistance for introducing the catheter 72 into the vasculature of the patient 70 and assists in reducing complications typically associated with such introduction, including inadvertent arterial puncture, hematoma, pneumothorax, etc.

The probe 40 includes a head that houses a piezoelectric array for producing ultrasonic pulses and for receiving echoes thereof after reflection by the patient's body when the head is placed against the patient's skin proximate a prospective insertion site (see FIG. 2). The probe 40 further includes a plurality of control buttons, which can be included on a button pad as shown in FIG. 2. The mode of the integrated system 10 can be controlled by the control buttons, thus eliminating the need for the clinician to reach out of the sterile field, which is established about the insertion site 73 prior to placement of the catheter 72, to change modes via use of the console button interface 32.

As such, in some embodiments a clinician employs the first mode (i.e., the US mode) to determine a suitable insertion site for establishing vascular access, first with a needle or introducer, then with the catheter 72. The clinician can then seamlessly switch to the second mode (i.e., the TLS mode) by way of control button pushes on the control button pad of the probe 40 without having to reach out of the sterile field. The TLS mode can then be used to assist in advancement of the catheter 72 through the vasculature toward an intended destination.

FIG. 1 shows that the probe 40 further includes a button-and-memory controller 42 for governing control button and probe operation. The button-and-memory controller 42 can include non-volatile memory such as EEPROM in some embodiments. The button-and-memory controller 42 is in operable communication with a probe interface 44 of the console 20, which includes a piezo input/output component 44A for interfacing with the probe piezoelectric array and a button-and-memory input/output component 44B for interfacing with the button-and-memory controller 42.

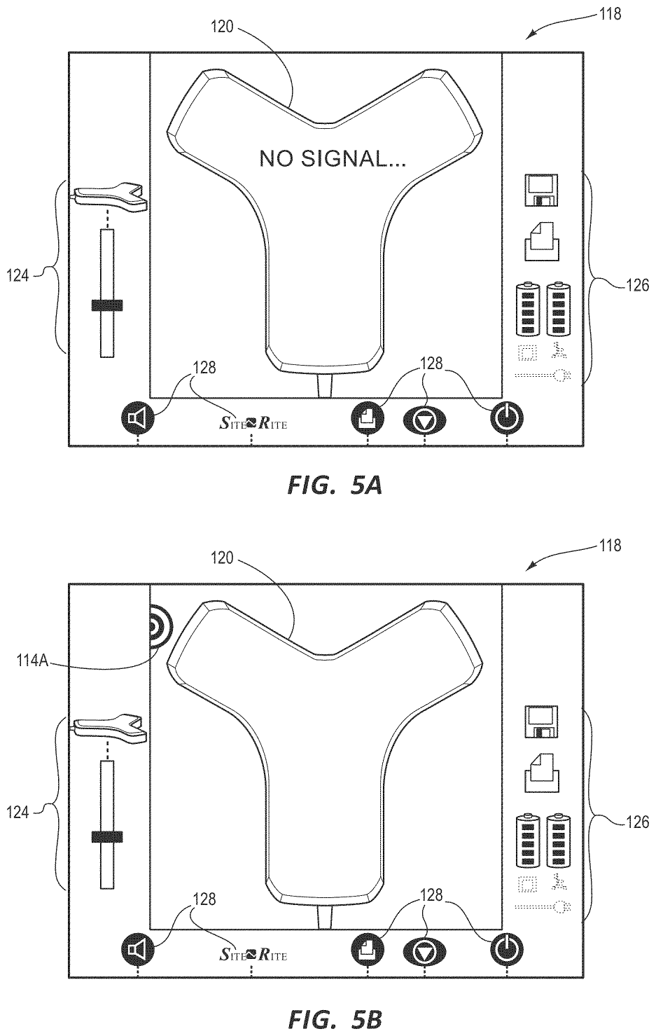

FIG. 3 shows an example screenshot 88 as depicted on the display 30 while the integrated system 10 is in its first mode (i.e., the US mode). An image 90 of a subcutaneous region of the patient 70 is shown depicting a cross section of a vein 92. The image 90 is produced by operation of the piezoelectric array of the probe 40. Also included on the screenshot 88 is a depth scale indicator 94, which provides information regarding the depth of the image 90 below the patient's skin; a lumen size scale 96 that provides information as to the size of the vein 92 relative to standard catheter lumen sizes; and other indicia 98 that provide information regarding status of the integrated system 10 or possible actions to be taken (e.g., freeze frame, image templates, data save, image print, power status, image brightness, etc.) with the integrated system 10.

Note that while the vein 92 is depicted in the image 90, other body lumens or portions can be imaged in other embodiments. Note that the US mode shown in FIG. 3 can be simultaneously depicted on the display 30 with other modes, such as the TLS mode, if desired. In addition to the display 30, aural information, such as beeps, tones, etc., can also be employed by the integrated system 10 to assist the clinician during placement of the catheter 72. Moreover, the control buttons included on the probe 40 and the console button interface 32 can be configured in a variety of ways for user or clinician input. Indeed, slide switches, toggle switches, electronic or touch-sensitive pads, or the like can be implemented for the user or clinician input. Additionally, both US visualization and TLS tracking can occur simultaneously or exclusively during use of the integrated system 10.

The probe 40 can be employed as part of the integrated system 10 to enable US visualization of the peripheral vasculature of the patient 70 in preparation for percutaneous introduction of the catheter 72. However, the probe 40 can also be employed to control functionality of the TLS mode of the integrated system 10 when navigating the catheter 72 toward its desired destination within the vasculature. Again, as the probe 40 is used within the sterile field of the patient 70, this feature enables TLS tracking to be controlled entirely from within the sterile field. Thus, the probe 40 is a dual-purpose device, enabling convenient control of both US visualization and TLS tracking of the integrated system 10 from the sterile field. In some embodiments, the probe 40 can also be employed to control some or all ECG-related functionality for the third mode of the integrated system 10.

The integrated system 10 further includes the second mode, namely the TLS mode. The TLS 50 enables the clinician to quickly locate and confirm the position or orientation of the catheter 72 during initial placement into and advancement through the vasculature of the patient 70. Specifically, the TLS mode detects a magnetic field generated by a magnetic field-producing tip of a stylet 100, which is pre-loaded in some embodiments into a longitudinally defined lumen of the catheter 72, thus enabling the clinician to ascertain the general location and orientation of the distal tip 76A of the catheter within the patient's body for tracking. In some embodiments, the magnetic field-producing tip of the stylet 100 can be tracked using the teachings of one or more patents of U.S. Pat. Nos. 5,775,322; 5,879,297; 6,129,668; 6,216,028; and 6,263,230, each of which is incorporated by reference in its entirety into this application. The TLS 50 also enables display of the direction in which the distal tip 76A of the catheter 72 is pointing, thus further assisting accurate placement of the catheter 72. The TLS 50 further assists the clinician in determining when a malposition of the distal tip 76A of the catheter 72 has occurred, for example, where the distal tip 76A has deviated from a desired venous path into another vein. Examples of the TLS 50 and systems incorporating the TLS 50 are disclosed in U.S. Pat. Nos. 8,388,541; 8,781,555; 8,849,382; 9,636,031; and 9,649,048, each of which is incorporated by reference in its entirety into this application.

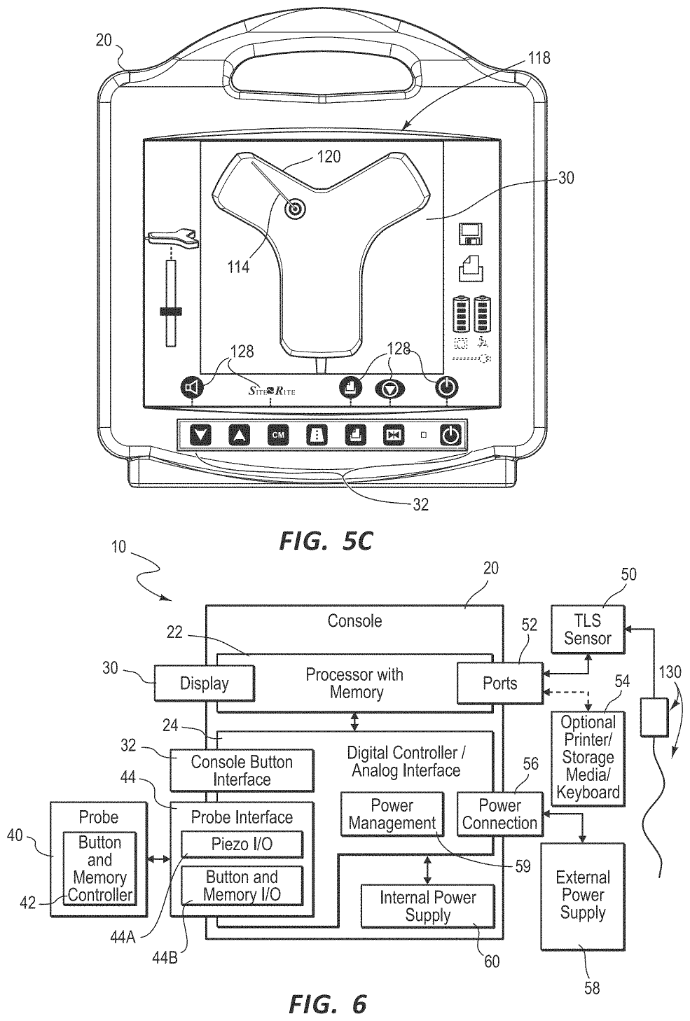

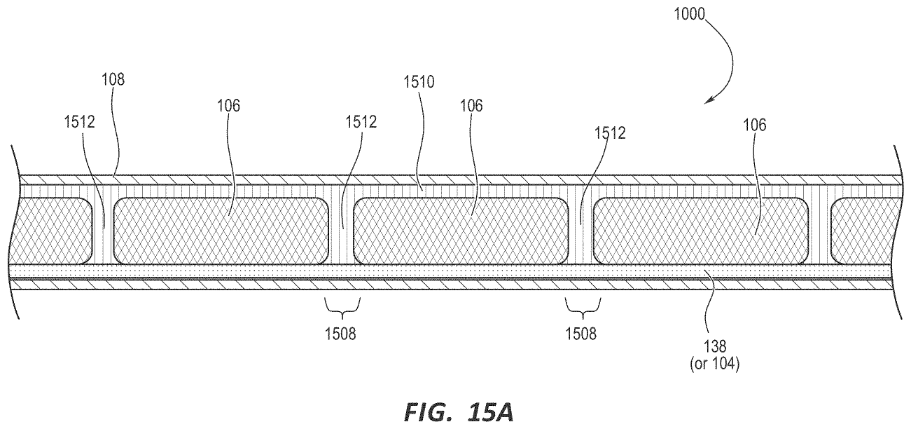

As mentioned, the TLS 50 utilizes the stylet 100 to enable the distal tip 76A of the catheter 72 to be tracked during its advancement through the vasculature. FIG. 4 gives an example of the stylet 100, which includes a proximal end 100A and a distal end 100B. A handle 102 is included over the stylet body 1000 (see FIG. 11) at the proximal end 100A of the stylet 100 with a core wire 104 distally extending from the handle 102 through the stylet body 1000. A magnetic assembly 1002 is distal of the core wire 104 or alongside the core wire 104 as shown in FIG. 11. The magnetic assembly 1002 includes one or more magnetic field-producing elements 106. For example, a plurality of the magnetic field-producing elements 106 can be disposed adjacent one another proximate the distal end 100B of the stylet 100 and encapsulated by a flexible outer construction 108 (e.g., a flexible casing) as shown in FIG. 4. Indeed, each magnetic field-producing element of the one-or-more magnetic field-producing elements 106 can include a solid, cylindrically shaped permanent magnet (e.g., composite or ferrite magnet, rare-earth magnet, rare-earth-free magnet, etc.) stacked end-to-end with the other magnetic field-producing elements 106. A stylet tip 110 can be sealed with a filling 1004 (e.g., a conductive slug, an adhesive such as a conductive epoxy, etc.) in a distal tip of the outer construction 108 distal of the one-or-more magnetic field-producing elements 106, thereby sealing the one-or-more magnetic field-producing elements 106 in the distal portion of the stylet 100 of the stylet body 1000 thereof. (See FIG. 11.) Advantageously, the one-or-more magnetic field-producing elements 106 can thusly move relative to the outer construction 108, which enhances flexibility of the stylet 100 over adhering the one-or-more magnetic field-producing elements 106 directly to the outer construction 108. Indeed, adhering the one-or-more magnetic field-producing elements 106 directly to the outer construction 108 keeps the one-or-more magnetic field-producing elements 106 in fixed in position relative to the outer construction 108.

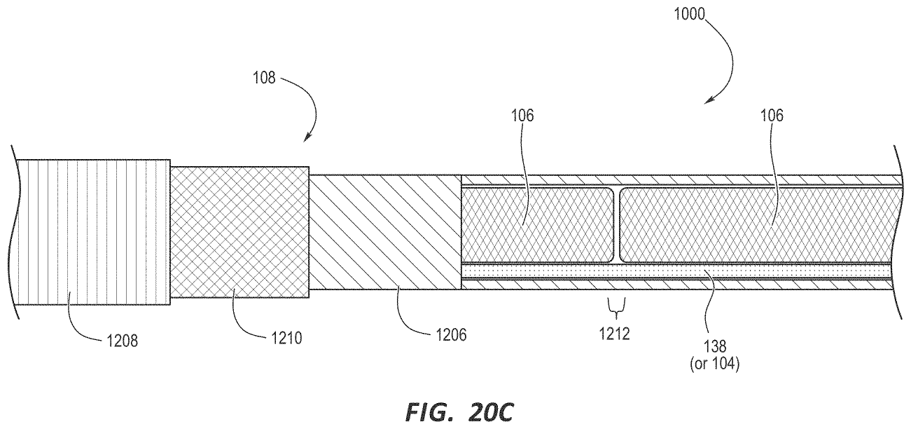

Notwithstanding the foregoing, the one-or-more magnetic field-producing elements 106 can vary from that set forth above with respect to number, shape, size or one or more dimensions, composition, type of magnet, or position in the distal portion of the stylet 100. Indeed, other examples of the one-or-more magnetic field-producing elements 106 are set forth below. For example, the magnetic assembly 1002 or the one-or-more magnetic field-producing elements 106 thereof can be a flexible electromagnetic assembly 1006 such as that set forth below with respect to FIG. 19, which produces a magnetic field for detection by the TLS 50. Another example of a magnetic assembly usable here can be found in U.S. Pat. No. 5,099,845, titled “Medical Instrument Location Means,” which is incorporated by reference in its entirety into this application. Yet other examples of stylets including magnetic assemblies that can be employed with the TLS 50 include U.S. Pat. Nos. 8,784,336; 9,901,714; 10,004,875; U.S. 2018/0304043; and U.S. 2018/0169389, each of which is incorporated by reference in its entirety into this application. It should be appreciated that “stylet,” as used herein, can include any one of a variety of devices configured for removable placement within a lumen of the catheter 72 to assist in placing the distal tip 76A of the catheter 72 in a desired location within the patient's vasculature.

FIG. 2 shows disposal of the stylet 100 substantially within a lumen in the catheter 72 such that the proximal portion of the stylet 100 extends proximally from the lumen of the catheter tube 71, through the hub 74A, and out through an extension leg of the one-or-more extension legs 74B. Disposed within the lumen of the catheter 72 as such, the distal end 100B of the stylet 100 is substantially co-terminal with the distal tip 76A of the catheter 72 such that detection by the TLS 50 of the distal end 100B or the stylet 100 correspondingly indicates the location of the distal tip 76A of the catheter 72.

The TLS 50 is employed by the integrated system 10 during operation to detect a magnetic field produced by the one-or-more magnetic field-producing elements 106 of the stylet 100. As seen in FIG. 2, the TLS 50 is placed on the chest of the patient 70 during insertion of the catheter 72. The TLS 50 is placed on the chest of the patient 70 in a predetermined location, such as through the use of external body landmarks, to enable the magnetic field of the one-or-more magnetic field-producing elements 106 disposed in the catheter 72 to be detected during transit of the catheter 72 through the patient's vasculature. Again, as the one-or-more magnetic field-producing elements 106 of the magnetic assembly 1002 can be co-terminal with the distal tip 76A of the catheter 72 (see FIG. 2). Detection by the TLS 50 of the magnetic field of the one-or-more magnetic field-producing elements 106 provides information to the clinician as to the position and orientation of the distal tip 76A of the catheter 72 during its transit.

In greater detail, the TLS 50 is operably connected to the console 20 of the integrated system 10 via one or more of the ports 52 as shown in FIG. 1. Note that other connection schemes between the TLS 50 and the console 20 can also be used without limitation. As just described, the one-or-more magnetic field-producing elements 106 are employed in the stylet 100 to enable the position of the distal tip 76A of the catheter 72 (see FIG. 2) to be observed relative to the TLS 50 placed on the patient's chest. Detection by the TLS 50 of the one-or-more magnetic field-producing elements 106 is graphically displayed on the display 30 of the console 20 during the TLS mode. In this way, a clinician placing the catheter 72 is able to generally determine the location of the distal tip 76A of the catheter 72 within the patient's vasculature relative to the TLS 50 and detect when malposition of the catheter 72, for example, advancement of the catheter 72 along an undesired vein, is occurring.

FIGS. 5A-5C depict screenshots taken from the display 30 of the integrated system 10 while in the TLS mode, showing how the magnetic assembly 1002 of the stylet 100 is depicted. The screenshot 118 of FIG. 5A shows a representative image 120 of the TLS 50. Other information is provided on the screenshot 118, including a depth scale indicator 124, status or action indicia 126, and button icons 128 corresponding to the console button interface 32 included on the console 20 (see FIG. 5C). Though the button icons 128 are shown as relatively simple indicators to guide the user in identifying the purpose of the corresponding buttons of the console button interface 32, in some embodiments the display 30 can be made touch-sensitive so that the button icons 128 themselves can function as button interfaces and change according to the mode of the integrated system 10.

During initial stages of advancement of the catheter 72 through the patient's vasculature after insertion therein, the distal tip 76A of the catheter 72, having the distal end 100B of the stylet 100 substantially co-terminal therewith, is relatively distant from the TLS 50. As such, the screenshot 118 indicates “no signal,” indicating that the magnetic field from the magnetic assembly 1002 of the stylet 100 has not been detected. In FIG. 5B, the magnetic assembly 1002 proximate the distal end 100B of the stylet 100 has advanced sufficiently close to the TLS 50 to be detected thereby, though it is not yet under the TLS 50. This is indicated by a half-icon 114A representing the distal portion of the stylet 100 or the magnetic assembly 1002 of the stylet 100 being positioned to the right of the TLS 50 from the perspective of the patient 70.

In FIG. 5C, the magnetic assembly 1002 proximate the distal end 100B of the stylet 100 has advanced under the TLS 50 such that its position and orientation relative thereto is detected by the TLS 50. This is indicated by an icon 114 on the image 120. Note that the button icons 128 provide indications of the actions that can be performed by pressing the corresponding buttons of the console button interface 32. As such, the button icons 128 can change in accordance with the mode of the integrated system 10, thereby providing flexibility of use for the console button interface 32. Note further that, as the control button pad of the probe 40 includes control buttons that mimic several of the buttons of the console button interface 32, the button icons 128 on the display 30 provide a guide to the clinician for controlling the integrated system 10 with the control buttons of the probe 40 while remaining in the sterile field. For instance, if the clinician has a need to leave the TLS mode and return to the US mode, the appropriate control buttons on the control button pad of the probe can be pressed, and the US mode can immediately be called up with the display 30 refreshing to accommodate the visual information needed for the US mode, such as that shown in FIG. 3. This is accomplished without a need for the clinician to reach out of the sterile field.

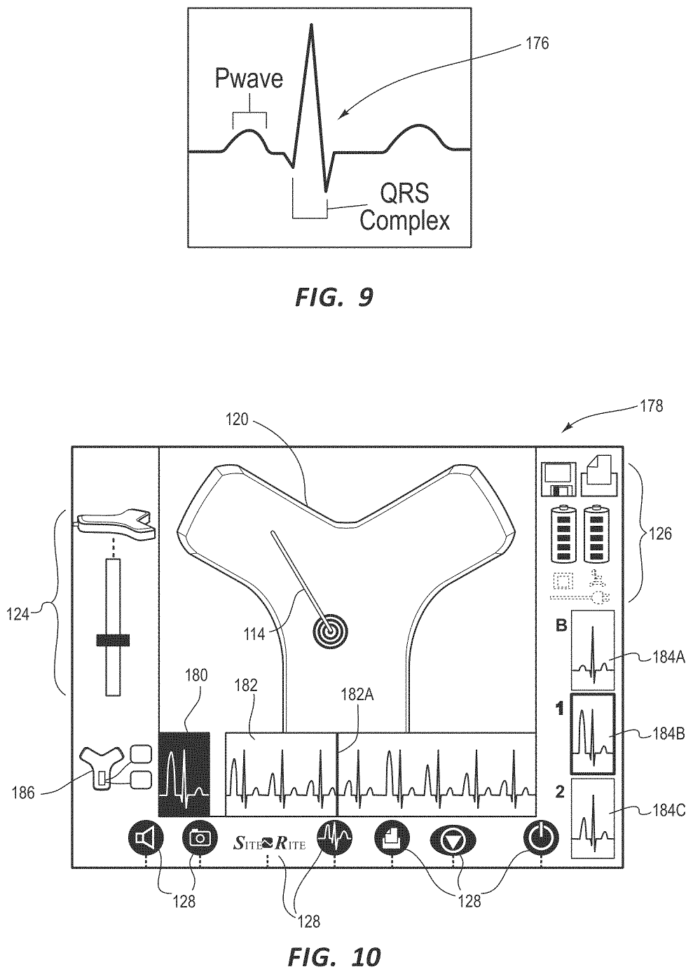

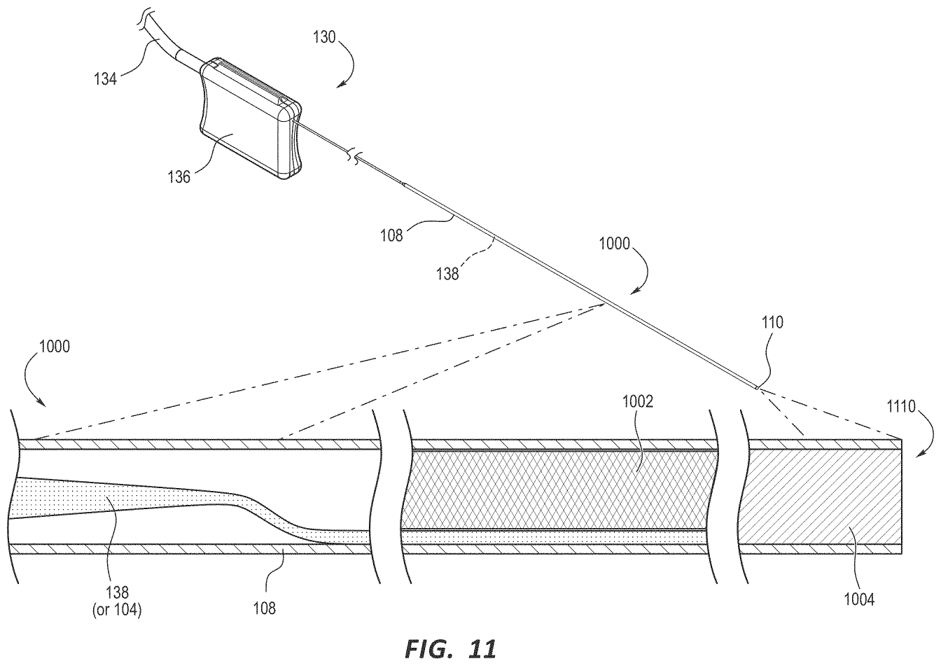

Reference is now made to FIGS. 6 and 7 in describing the integrated system 10 according to some embodiments. As before, the integrated system 10 includes the console 20, the display 30, the probe 40 for US visualization, and the TLS 50 for TLS tracking. Note that the integrated system 10 depicted in FIGS. 6 and 7 is similar in many respects to the integrated system 10 shown in FIGS. 1 and 2. As such, only selected differences are described below. The integrated system 10 of FIGS. 6 and 7 includes additional functionality for determination of the proximity of the distal tip 76A of the catheter 72 relative to a sino-atrial (“SA”) or other electrical impulse-emitting node of the heart of the patient 70, thus providing enhanced ability to accurately place the distal tip 76A of the catheter 72 in a desired location proximate the node. Also, the third mode or ECG mode of the integrated system 10 enables detection of ECG signals from the SA node in order to place the distal tip 76A of the catheter 72 in a desired location within the patient's vasculature. Note that the US mode, the TLS mode, and the ECG mode are seamlessly combined in the integrated system 10 of FIG. 6 and can be employed in concert or individually to assist in placement of the catheter 72.

FIGS. 6 and 7 show the addition to the integrated system 10 of a stylet 130. As an overview, the stylet 130 is removably predisposed within the lumen of the catheter 72 being inserted into the patient 70 via the insertion site 73. In addition to the magnetic assembly 1002 of the stylet 100 for the TLS mode, the stylet 130 includes an ECG-sensor assembly proximate its distal end 130B for sensing ECG signals produced by the SA node. In contrast to the stylet 100, the stylet 130 further includes a tether 134 extending from its proximal end that operably connects to the TLS 50. The tether 134 permits the ECG signals detected by the ECG-sensor assembly of the stylet 130 to be conveyed to the TLS 50 during confirmation of the location of the distal tip 76A of the catheter 72 as part of the ECG mode. As shown in FIG. 7, reference and ground ECG leads attach to the body of the patient 70 and to the TLS 50, thereby enabling the integrated system 10 to filter out high-level electrical activity unrelated to the electrical activity of the SA node of the heart, which, in turn, enables ECG-based tip confirmation. Together with the reference and ground signals received from the ECG leads placed on the patient's skin, the ECG signals sensed by the ECG-sensor assembly of the stylet 130 are received by the TLS 50 positioned on the patient's chest (see FIG. 7). The TLS 50 or processor 22 can process ECG data corresponding to the ECG signals to produce an ECG waveform on the display 30. In the case where the TLS 50 processes the ECG data, a processor is included therein to perform the intended functionality. If the console 20 processes the ECG data, the processor 22, the digital controller/analog interface 24, or other processor can be utilized in the console 20 to process the data.

Thus, as it is advanced through the patient's vasculature, the catheter 72 equipped with the stylet 130 can advance under the TLS 50, which is positioned on the chest of the patient 70 as shown in FIG. 7. This enables the TLS 50 to detect the position of the magnetic assembly 1002 of the stylet 130, which is substantially co-terminal with the distal tip 76A of the catheter 72 as located within the patient's vasculature. The detection by the TLS 50 of the magnetic assembly 1002 of the stylet 130 is depicted on the display 30 during the ECG mode. The display 30 further depicts during the ECG mode an ECG waveform produced as a result of the patient's cardiac electrical activity as detected by the ECG-sensor assembly of the stylet 130. In greater detail, the electrical activity of the SA node, including the P-wave of the waveform, is detected by the ECG-sensor assembly of the stylet 130 and forwarded to the TLS 50 and console 20. The electrical activity of the SA node is then processed for depiction on the display 30. The clinician placing the catheter 72 can then observe the ECG data to determine optimum placement of the distal tip 76A of the catheter 72 such as proximate the SA node. In some embodiments, the console 20 includes the electronic components such as the processor 22 (see FIG. 6) necessary to receive and process the signals detected by the ECG-sensor assembly of the stylet 130. However, in some embodiments, the TLS 50 can include the electronic components necessary to receive and process the signals detected by the ECG-sensor assembly of the stylet 130.

As already discussed, the display 30 is used to display information to the clinician during the placement of the catheter 72. The content of the display 30 changes according to the mode of the integrated system 10, namely the US mode, the TLS mode, the ECG mode, or any combination of the foregoing modes. Any of the three modes can be immediately called up to the display 30 by the clinician, and, in some cases, information from multiple modes, such as the TLS and ECG modes, can be displayed simultaneously. In some embodiments, as before, the mode of the integrated system 10 can be controlled by the control buttons of the probe 40, thus eliminating the need for the clinician to reach out of the sterile field to touch the console button interface 32 of the console 20 to change modes. Thus, the probe 40 can be employed to also control some or all ECG-related functionality of the integrated system 10. Note that the console button interface 32 or other input configurations can also be used to control system functionality. Also, in addition to the display 30, aural information, such as beeps, tones, etc., can also be employed by the integrated system 10 to assist the clinician during placement of the catheter 72.

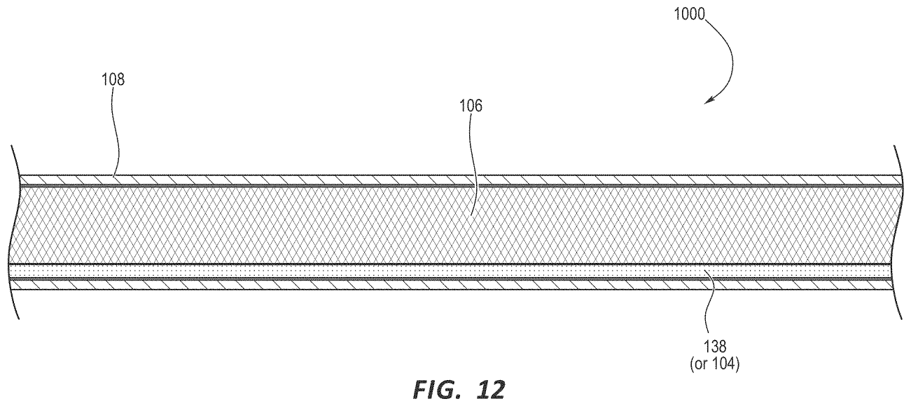

Reference is now made to FIG. 8 in describing various details of some embodiments of the stylet 130 that is removably loaded into the catheter 72 and employed during insertion to position the distal tip 76A of the catheter 72 in a desired location within the patient's vasculature. As shown, the stylet 130 includes a proximal end 130A and a distal end 130B. A tether connector 132 is included at the proximal end 130A of the stylet 130, and the tether 134 extends distally from the tether connector 132 and attaches to a handle 136. A core wire 138 extends distally from the handle 136. In some embodiments, the stylet 130 is pre-loaded within a lumen of the catheter 72 such that the distal end 130B is substantially flush, or co-terminal, with the opening at the distal tip 76A of the catheter 72 (see FIG. 7). In addition, a proximal portion of the core wire 138, the handle 136, and the tether 134 extend proximally from an extension leg of the one-or-more extension legs 74B in such embodiments. Note that, though described herein as a stylet, in other embodiments a guidewire or other medical-device guiding apparatus could include certain working features of the stylet 130.

The core wire 138 defines an elongate shape and is composed of a suitable stylet material including stainless steel or a memory material such as a nickel and titanium-containing alloy commonly known as “nitinol.” Though not shown here, manufacture of the core wire 138 from nitinol enables the portion of the core wire 138 corresponding to a distal segment of the stylet 130 to have a pre-shaped (e.g., bent) configuration so as to urge a distal portion 76 of the catheter 72 into a similar configuration. In other embodiments, the core wire 138 includes no pre-shaping. Further, the nitinol construction lends torqueability to the core wire 138 to enable at least a distal segment of the stylet 130 to be manipulated by the core wire 138 while the stylet 130 is disposed within the lumen of the catheter 72, which, in turn, enables the distal portion 76 of the catheter 72 to be navigated through the vasculature during insertion of the catheter 72.

The handle 136 is provided to enable insertion or removal of the stylet 130 from the catheter 72. In embodiments where the core wire 138 is torqueable, the handle 136 further enables the core wire 138 to be rotated within the lumen of the catheter 72 to assist in navigating the distal portion 76 of the catheter 72 through the vasculature of the patient 70.

The handle 136 attaches to a distal end of the tether 134. The tether 134, in turn, can be a flexible, shielded cable housing one or more conductive wires electrically connected both to the core wire 138, which acts as the ECG-sensor assembly, and the tether connector 132. As such, the tether 134 provides a conductive pathway from the distal portion of the core wire 138 through to the tether connector 132 at proximal end 130A of the stylet 130. The tether connector 132 is configured for operable connection to the TLS 50 on the patient's chest for assisting in navigation of the distal tip 76A of the catheter 72 to a desired location within the patient's vasculature.

As set forth above for the stylet 100, the outer construction 108 (e.g., the casing) encapsulates at least a portion of the core wire 138 as well as the magnetic assembly 1002 disposed proximate the distal end 130B of the stylet 130 for use during the TLS mode of the integrated system 10. The magnetic assembly 1002 includes the one-or-more magnetic field-producing elements 106, which can be interposed between an outer surface of core wire 138 and an inner surface of the outer construction 108 proximate the distal end 130B of the stylet 130. The one-or-more magnetic field-producing elements 106 can include up to at least 20 permanent magnets of a solid cylindrical shape stacked end-to-end in a manner similar to the stylet 100 of FIG. 4. In other embodiments, however, the one-or-more magnetic field-producing elements 106 can vary in number, shape, size or one or more dimensions, composition, type of magnet, or position in the distal portion of the stylet 130. For example, in some embodiments, for example, the one-or-more magnetic field-producing elements 106 of the magnetic assembly 1002 is replaced with an electromagnet that produces a magnetic field for detection by the TLS 50.

The one-or-more magnetic field-producing elements 106 are employed in the stylet 130 distal portion to enable the position of the distal end 130B of the stylet 130 to be observable relative to the TLS 50 placed on the patient's chest. As set forth above, the TLS 50 is configured to detect a magnetic field produced by the one-or-more magnetic field-producing elements 106 as the stylet 130 advances with the catheter 72 through the patient's vasculature. In this way, a clinician placing the catheter 72 is generally able to determine the location of the distal tip 76A of the catheter 72 within the patient's vasculature and detect when malposition of the catheter 72 is occurring.

The stylet 130 further includes the aforementioned ECG-sensor assembly. The ECG-sensor assembly enables the stylet 130, disposed in a lumen of the catheter 72 during insertion, to be employed in detecting an intra-atrial ECG signals produced by an SA or other node of the patient's heart, thereby allowing for navigation of the distal tip 76A of the catheter 72 to a predetermined location within the vasculature proximate the patient's heart. Thus, the ECG-sensor assembly serves as an aide in confirming proper placement of the distal tip 76A of the catheter 72.

In the embodiment illustrated in FIG. 8, the ECG-sensor assembly includes a distal portion of the core wire 138 disposed proximate the distal end 130B of the stylet 130. The core wire 138, being electrically conductive, enables the ECG signals to be detected by the distal end thereof and transmitted proximally along the core wire 138. The filling 1004 (e.g., a metal slug or a metal particle-containing epoxy) can fill a distal portion of the outer construction 108 including the stylet tip 110 (see FIG. 4) adjacent the distal termination of the core wire 138 so as to be in conductive communication with the distal end of the core wire 138. This in turn increases the conductive surface of the distal end 130B of the stylet 130 so as to improve its ability to detect the ECG signals.

Before placement of the catheter 72, the stylet 130 is loaded into a lumen of the catheter 72. Note that the stylet 130 can come preloaded in the catheter 72 from the manufacturer or loaded into the catheter 72 by a clinician prior to placing the catheter 72. The stylet 130 is disposed within the catheter 72 such that the distal end 130B of the stylet 130 is substantially co-terminal with the distal tip 76A of the catheter 72, thus placing the distal tips of both the stylet 130 and the catheter 72 in substantial alignment with one another. The co-terminality of the catheter 72 and stylet 130 enables the magnetic assembly 1002 to function with the TLS 50 in the TLS mode for tracking the position of the distal tip 76A of the catheter 72 as it advances within the patient's vasculature. For the tip confirmation functionality of the integrated system 10, however, the distal end 130B of the stylet 130 need not be co-terminal with the distal tip 76A of the catheter 72. Rather, all that is required is that a conductive path between the vasculature and the ECG-sensor assembly of the core wire 138 be established such that electrical impulses of the SA node or other node of the patient's heart can be detected. This conductive path can include various components including saline solution, blood, etc.

Once the catheter 72 has been introduced into the patient's vasculature via the insertion site 73 (see FIG. 7) the TLS mode of the integrated system 10 can be employed as already described to advance the distal tip 76A of the catheter 72 toward its intended destination proximate the SA node. Upon approaching the foregoing destination, the integrated system 10 can be switched to the ECG mode to enable the ECG signals emitted by the SA node to be detected. As the stylet-loaded catheter 72 is advanced toward the patient's heart, the electrically conductive ECG-sensor assembly, including the distal end of the core wire 138 and the conductive material in the stylet tip 110, begins to detect the electrical impulses produced by the SA node. As such, the ECG-sensor assembly serves as an electrode for detecting the ECG signals. The core wire 138 proximal to the distal end of the stylet 130 serves as a conductive pathway to convey the electrical impulses produced by the SA node and received by the ECG-sensor assembly to the tether 134.

The tether 134 conveys the ECG signals to the TLS 50 temporarily placed on the patient's chest. The tether 134 is operably connected to the TLS 50 via the tether connector 132 or other suitable direct or indirect connections. As described, the ECG signals can then be processed and depicted on the display 30 (see FIGS. 6 and 7). Monitoring the ECG signals received by the TLS 50 and displayed on the display 30 enables a clinician to observe and analyze changes in the ECG signals as the distal tip 76A of the catheter 72 advances toward the SA node. When the ECG signals received match a desired profile, the clinician can determine that the distal tip 76A of the catheter 72 has reached a desired position with respect to the SA node. As mentioned, in some embodiments this desired position lies within the lower one-third portion of the SVC.

The ECG-sensor assembly and the magnetic assembly 1002 can work in concert in assisting a clinician in placing the catheter 72 within a patient's vasculature. Generally, the magnetic assembly 1002 of the stylet 130 assists the clinician in generally navigating the vasculature from initial insertion of the catheter 72 to placing the distal tip 76A of the catheter 72 in a desired general region of the patient's heart. The ECG-sensor assembly can then be employed to guide the distal tip 76A of the catheter 72 to the desired location within the SVC by enabling the clinician to observe changes in the ECG signals produced by the patient's heart as the ECG-sensor assembly of the stylet 130 approaches the SA node. Again, once the ECG signals match a desired profile, the clinician can determine that the distal ends of both the stylet 130 and the catheter 72 have arrived at the desired location with respect to the patient's heart. Once it has been positioned as desired, the catheter 72 can be secured in place and the stylet 130 removed from the catheter 72. It is noted here that the stylet 130 can include one of a variety of configurations in addition to what is explicitly described herein. In some embodiments, the stylet 130 can attach directly to the console 20 instead of an indirect attachment via the TLS 50. In some embodiments, the structure of the stylet 130 that enables its TLS and ECG-related functionalities can be integrated into the catheter 72 itself. For instance, the magnetic assembly 1002 or the ECG-sensor assembly can, in some embodiments, be incorporated into the wall of the catheter 72.

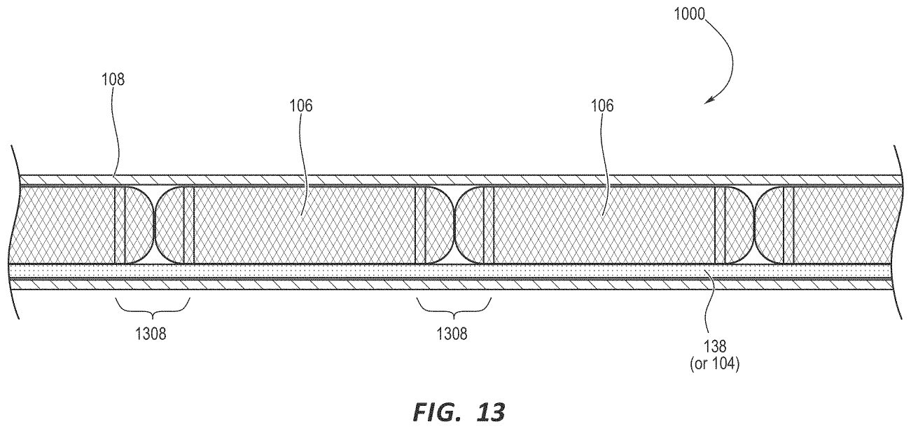

FIG. 9 shows a typical ECG waveform 176 including a P-wave and a QRS complex. Generally, the amplitude of the P-wave varies as a function of distance of the ECG-sensor assembly from the SA node, which produces the ECG waveform 176. A clinician can use this relationship in determining when the distal tip 76A of the catheter 72 is properly positioned proximate the heart. For instance, in one implementation, the distal tip 76A of the catheter 72 is desirably placed within the lower one-third (⅓^{rd}) of the superior vena cava. The ECG data detected by the ECG-sensor assembly of the stylet 130 is used to reproduce waveforms such as the ECG waveform 176 for depiction on the display 30 of the integrated system 10 during the ECG mode.

Reference is now made to FIG. 10 in describing display aspects of the ECG data on the display 30 when the integrated system 10 is in the ECG mode according to some embodiments. A screenshot 178 of the display 30 includes elements of the TLS mode such as the image 120 of the TLS 50 and the icon 114 corresponding to the position of the distal end 130B of the stylet 130 during transit through the patient's vasculature. The screenshot 178 further includes a window 180 in which the current ECG waveform captured by the ECG-sensor assembly of the stylet 130 is displayed. The window 180 is continually refreshed as new waveforms are detected.

Window 182 includes a successive depiction of the most recently detected ECG waveforms as well as a refresh bar 182A, which moves laterally to refresh the waveforms as they are detected. For comparison purposes, window 184A is used to display a baseline ECG waveform captured before the ECG-sensor assembly is brought into proximity with the SA node to assist the clinician in determining when the desired location of the distal tip 76A of the catheter 72 has been achieved. Windows 184B and 184C can be filled by user-selected ECG waveforms from those detected when the user pushes a predetermined control button on the probe 40 or the console button interface 32. The waveforms in the windows 184B and 184C remain until overwritten by new waveforms as a result of user selection via button pushes or other input. The depth scale indicator 124, status or action indicia 126, and button icons 128 are included on the display 30 as well. An integrity indicator 186 is also included on the display 30 to give an indication of whether the references and ground ECG leads are operably connected to the TLS 50.

The display 30 therefore depicts, in some embodiments, elements of both the TLS and ECG modes simultaneously on a single screen, thus offering the clinician ample data to assist in placing the distal tip 76A of the catheter 72 in a desired position. Note further that the screenshot 178 or selected ECG or TLS data can be saved, printed, or otherwise preserved by the integrated system 10 to enable documentation of proper placement of the catheter 72.

FIGS. 4 and 8 respectively illustrate the stylets 100 and 130 in accordance with some embodiments. FIG. 11 illustrates the stylet body 1000 of the stylets 100 and 130 in accordance with some embodiments.

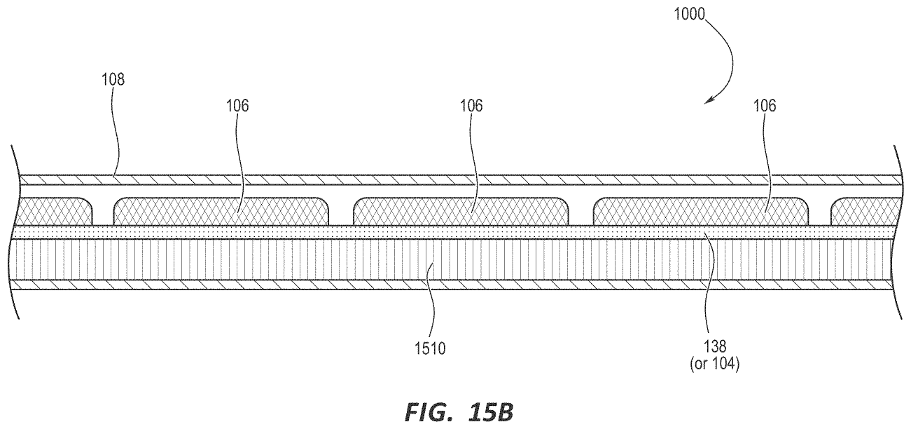

As shown, each magnetically trackable stylet of the stylets 100 and 130 includes a stylet body 1000 configured to be disposed in a lumen of a medical device such as the one-or-more lumens of the catheter 72 for magnetically tracking a tip of the medical device in vivo. The stylet body 1000 generally includes the core wire 104 or 138, the magnetic assembly 1002 including the one-or-more magnetic field-producing elements 106, and the outer construction 108. Again, the core wire 104 or 138 can be disposed alongside the one-or-more magnetic field-producing elements 106 in the distal portion of the stylet body 1000, thereby enabling magnetic tracking of the stylet 100 or 130. However, the core wire 104 or 138 can be alternatively disposed through the one-or-more magnetic field-producing elements 106 (e.g., through an axial center of the one-or-more magnetic field-producing elements 106) in the distal portion of the stylet body 1000. Notably, these are different configurations than that set forth above with respect to FIG. 4 where the one-or-more magnetic field-producing elements 106 are disposed distal of the core wire 104 or 138. No matter how the core wire 104 or 138 is disposed among the one-or-more magnetic field-producing elements 106, the core wire 104 or 138 can be tapered in the distal portion of the stylet body 1000 as shown in FIG. 11 to allow the one-or-more magnetic field-producing elements 106 to be sized as needed. While certain embodiments of the magnetic assembly 1002, the outer construction 108 of the stylet body 1000, or the like are set forth above for the stylets 100 and 130, additional embodiments of at least the magnetic assembly 1002 and the outer construction 108 of the stylet body 1000 are respectively set forth below with respect to FIGS. 12-14, 15A, 15B, and 16-19 and FIGS. 20A-20C, 21A, and 21B.

FIG. 12 illustrates a detailed cross-sectional view of the distal portion of the stylet 100 or 130 in accordance with some embodiments.

The one-or-more magnetic field-producing elements 106 can include one or more polymer-bonded magnets. The one-or-more polymer-bonded magnets can include a single polymer-bonded magnet molded into a cylinder. The one-or-more polymer-bonded magnets can alternatively include a plurality of polymer-bonded magnets molded into cylinders. Such polymer-bonded magnets can include, but are not limited to, polymer-bonded neodymium magnets. The one-or-more polymer-bonded magnets are configured to enhance flexural ability of the stylet body 1000 and, hence, the stylet 100 or 130. Indeed, the one-or-more polymer-bonded magnets are configured to bend and, thus, allow the stylet body 1000 to bend in accordance with an anatomy (e.g., a vasculature) of a patient without kinking or breaking the stylet body 1000.

Advantageously, the shape, the dimension (e.g., the length), the materials (e.g., magnetic material, polymer, etc.), the magnetic saturation, or the loading of the single polymer-bonded magnet or each polymer-bonded magnet of the plurality of polymer-bonded magnets can be optimized to provide a desired balance between magnetic field strength of the magnetic assembly 1002 and flexibility (e.g., bending radius) of the distal portion of the stylet body 1000. In addition, the outer construction 108 can be optimized for overall support, tensile strength, and flexibility.

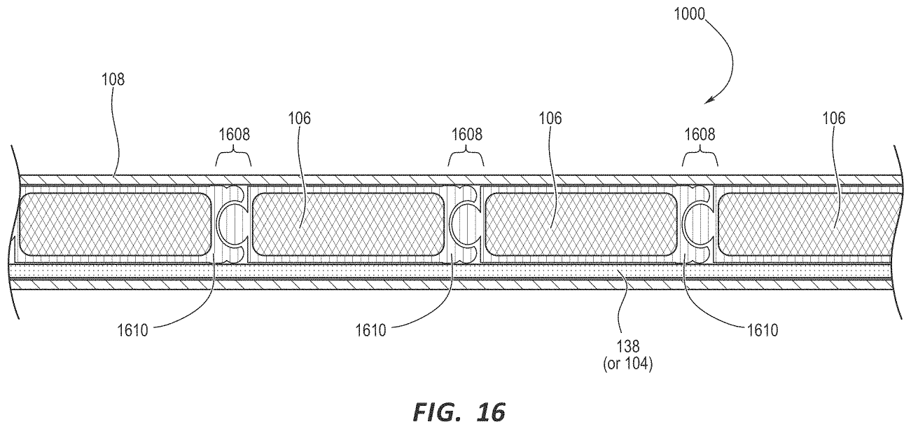

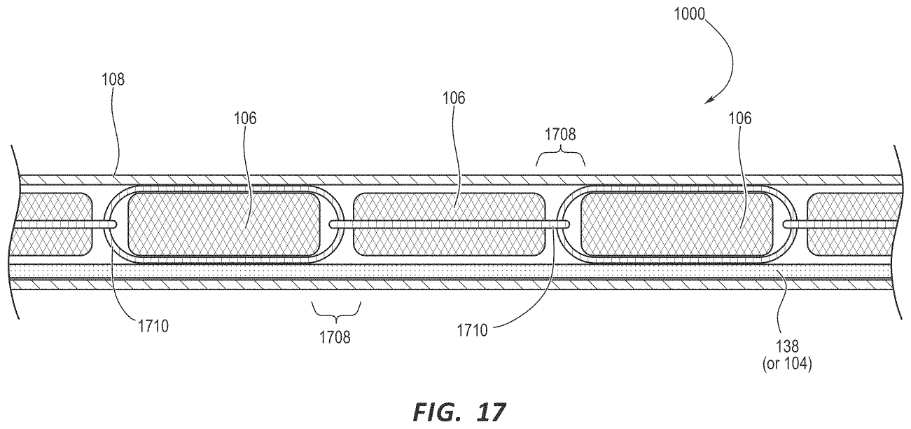

FIGS. 13 and 14 illustrate detailed cross-sectional views of the distal portion of stylet 100 or 130 in accordance with some embodiments.

The one-or-more magnetic field-producing elements 106 can include one or more sintered magnets. The one-or-more sintered magnets can include a plurality of sintered magnets cut and finished into cylinders or even cones having flat or radiused ends as shown in FIGS. 13 and 14. Optionally, the plurality of cylindrical or conical sintered magnets can alternate with a plurality of spherical sintered magnets 1406 or a plurality of non-metallic spheres 1410 and, hence, non-magnetic spheres, as shown in FIG. 14. Such sintered magnets can include, but are not limited to, sintered neodymium magnets. Whether the plurality of cylindrical or conical sintered magnets include only the radiused ends or the plurality of cylindrical or conical sintered magnets include the flat or radiused ends alternating with the plurality of spherical sintered magnets 1406 or non-metallic spheres 1410 (e.g., thermoplastic spheres, elastomeric spheres, etc.), the distal portion of the stylet body 1000 so configured includes articulable joints 1308 or 1408 between at least the cylindrical magnets for enhanced flexural ability of the stylet 100 or 130. Indeed, the joints 1308 or 1408 are configured to allow at least the cylindrical magnets to bend into each other. This, in turn, allows the stylet body 1000 to bend in accordance with an anatomy (e.g., a vasculature) of a patient without kinking or breaking the stylet body 1000.

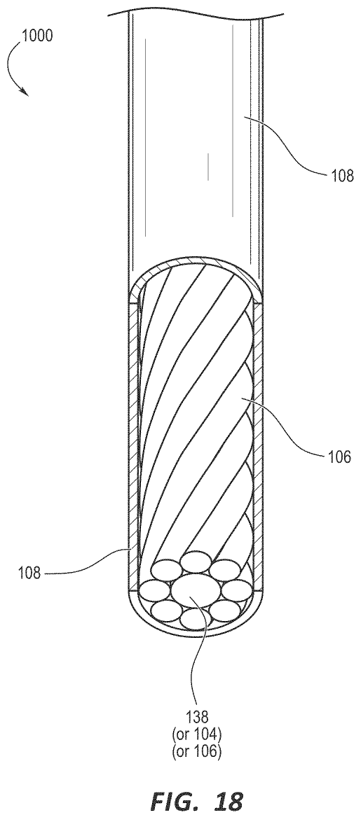

FIGS. 15A, 15B, 16, and 17 illustrate detailed cross-sectional views of the distal portion of stylet 100 or 130 in accordance with some embodiments.

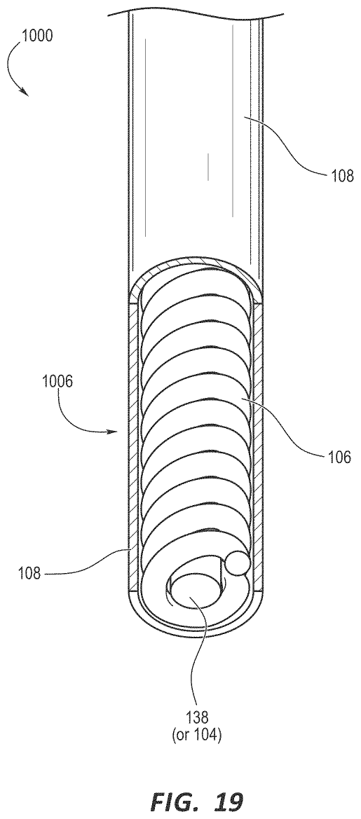

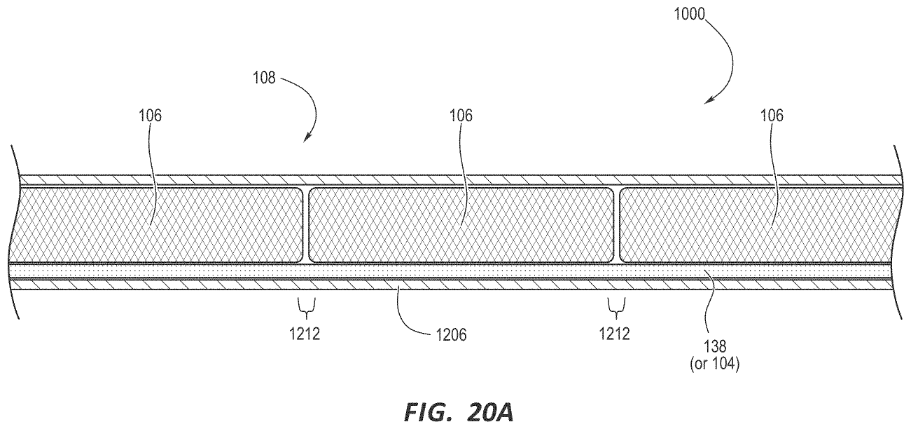

Again, the one-or-more magnetic field-producing elements 106 can include one or more sintered magnets; however, as shown in FIGS. 15A, 15B, 16, and 17, the one-or-more sintered magnets can be disposed in one or more magnet holders 1510, 1610, or 1710. The one-or-more sintered magnets can include a plurality of sintered magnets cut and finished into cylinders having flat or radiused ends. As shown in FIGS. 15A and 15B, the plurality of sintered magnets can be disposed in a groove of the single magnet holder 1510 including a plurality of septa 1512 disposed in the groove that section the groove of the magnet holder 1510. Indeed, the plurality of sintered magnets alternate with the plurality of septa 1512 in the groove of the magnet holder 1510, which septa form the articulable joints 1508 between the sintered magnets. As shown in FIGS. 16 and 17, the plurality of sintered magnets can be alternatively disposed in a plurality of magnet holders 1610 or 1710 forming the articulable joints 1608 or 1708 therebetween. As shown in FIG. 16, each magnet holder of the plurality of magnet holders 1610 can encase a sintered magnet of the plurality of sintered magnets. Further, each magnet holder of the plurality of magnet holders 1610 can include a ball end and a socket end such that the joints 1608 between the magnet holders 1610 are ball-and-socket joints. However, the plurality of magnet holders 1610 can include ball-ended magnet holders having a pair of ball ends alternating with socket-ended magnet holders having a pair of socket ends, which also provides ball-and-socket joints between the magnet holders 1610. As shown in FIG. 17, each magnet holder of the plurality of magnet holders 1710 can alternatively be a link or chain link and the joints 1708 can be interlinks between the links chained together. Whether the single magnet holder 1510 of FIGS. 15A and 15B or the plurality of magnet holders 1610 or 1710 of FIG. 16 or 17, the joints 1508, 1608, and 1708 enhance flexural ability of the stylet 100 or 130. Indeed, the joints 1508, 1608, and 1708 are configured to allow the plurality of sintered magnets to bend into each other and, thereby, allow the stylet 100 or 130 to bend in accordance with an anatomy of a patient without breaking.

Advantageously, the shape, the dimension (e.g., the length), the magnetic material, the magnetic saturation, or the loading of each sintered magnet of the plurality of cylindrical, conical, or spherical sintered magnets can be optimized to provide a desired balance between magnetic field strength of the magnetic assembly 1002 and flexibility (e.g., bending radius) of the distal portion of the stylet body 1000. Likewise, the loading or ratio of the plurality of non-metallic spheres 1410 to the plurality of cylindrical or conical sintered magnets can be optimized with to provide the desired balance between the magnetic field strength of the magnetic assembly 1002 and the flexibility (e.g., bending radius) of the distal portion of the stylet body 1000. In addition, the outer construction 108 can be optimized for overall support, tensile strength, and flexibility.

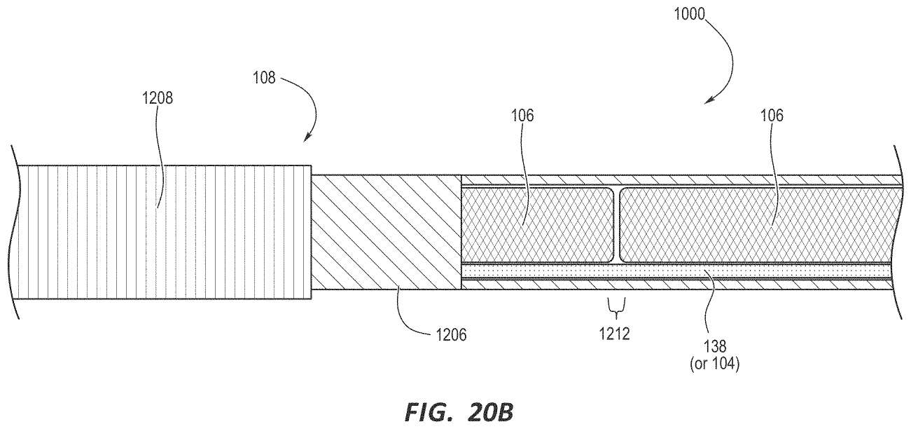

FIGS. 18 and 19 illustrate detailed cutaway views of the distal portion of the stylet 100 or 130 in accordance with some embodiments.

As shown, the one-or-more magnetic field-producing elements 106 include one or more magnetic wires around the core wire 104 or 138 if the core wire 104 or 138 is present. The one-or-more magnetic wires can include a single magnetic wire twisted with the core wire 104 or 138 or heliacally wrapped around the core wire 104 or 138 as shown in FIG. 19. The one-or-more magnetic wires can alternatively include a plurality of magnetic wires twisted or braided around i) the core wire 104 or 138, ii) a magnetic wire of the plurality of magnetic wires, or iii) each other around as shown in FIG. 18. Such magnetic wires can include, but are not limited to, wires of neodymium magnet. Being flexible, the one-or-more magnetic wires are configured to enhance flexural ability and allow bending of the stylet 100 or 130 in accordance with an anatomy (e.g., a vasculature) of a patient without kinking or breaking the stylet body 1000.

As alternatively shown in FIG. 19, the one-or-more magnetic field-producing elements 106 can include one or more electromagnets around the core wire 104 or 138. The one-or-more electromagnets can include a single electromagnet formed of a conductive wire heliacally wrapped around the core wire 104 or 138 for producing a magnetic field when supplied an electrical current. Alternatively, the one-or-more electromagnets can include a plurality of electromagnets formed of a plurality of conductive wires heliacally wrapped around the core wire 104 or 138 for producing a magnetic field when supplied an electrical current. Each conductive wire of the conductive wires can be heliacally wrapped around a dedicated section of the core wire 104 or 138 and electrically isolated from another conductive wire as well as the core wire 104 or 138 for a linear array of the plurality of electromagnets. Being flexible, the one-or-more conductive wires are configured to enhance flexural ability and allow bending of the stylet 100 or 130 in accordance with an anatomy (e.g., a vasculature) of a patient without kinking or breaking the stylet body 1000.

Advantageously, the dimension (e.g., the diameter, the length, etc.), the magnetic or conductive material (e.g., a same or a mixture of different magnetic or conductive materials for the plurality of magnetic or conductive wires), the magnetic saturation, the windings of the single conductive wire or the plurality of conductive wires, or the twisting or braiding of the plurality of magnetic or conductive wires can be optimized to provide a desired balance between magnetic field strength of the magnetic assembly 1002 and flexibility (e.g., bending radius) of the distal portion of the stylet body 1000. In addition, the outer construction 108 can be optimized for overall support, tensile strength, and flexibility.

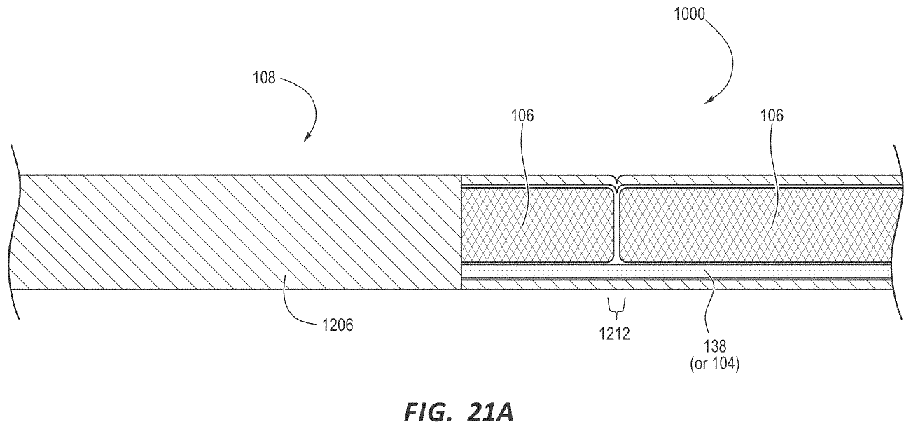

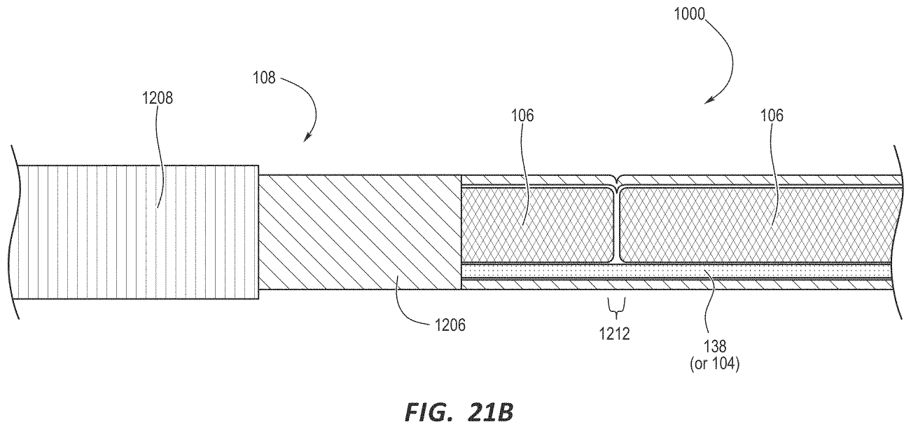

FIGS. 20A-20C, 21A, and 21B illustrate various detailed cross-sectional views of the distal portion of the stylet 100 or 130 including various outer constructions in accordance with some embodiments.

The outer construction 108 can be of a single layer (i.e., a single-layered outer construction) as shown in FIGS. 20A and 21A or multiple layers (i.e., a multi-layered outer construction) as shown in FIGS. 20B, 20C, and 21B. As to the single-layered outer construction 108, such an outer construction can include a primary layer 1206 of at least an overmolded layer, a reflowed layer, a potting layer, or a shrink-wrapped layer. As to the multi-layered outer construction 108, such an outer construction can include the primary layer 1206 together with one or more other layers such as a secondary layer 1208, a tertiary layer 1210, etc. thereover or thereunder. If the outer construction 108 includes two or more other layers in addition to the primary layer 1206 such as the secondary layer 1208 and the tertiary layer 1210, the two-or-more other layers can be in any combination of over, under, or over and under the primary layer 1206.

As to the primary layer 1206 being the overmolded layer, the overmolded layer can be molded around the core wire 104 or 138 and the magnetic assembly 1002 as shown in FIG. 20A. If present, one or more gaps between any two or more magnetic field-producing elements 106 can include polymeric material of the overmolded layer therebetween, thereby forming one or more articulable joints 1212 in the magnetic assembly 1002. The polymeric material can be an elastomer or a thermoplastic polymer such as acrylic, acrylonitrile butadiene styrene (“ABS”), polyamide (e.g., nylon), polylactic acid, polybenzimidazole, polycarbonate, polyether sulfone, polyoxymethylene, polyether ether ketone (“PEEK”), polyetherimide, polyethylene, polyphenylene oxide, polyphenylene sulfide, polypropylene, polystyrene, polyvinyl chloride, polyvinylidene fluoride, or polytetrafluoroethylene (“PTFE”). Because the one-or-more gaps between the two-or-more magnetic field-producing elements 106 can be adjusted with respect to their widths before molding (e.g., injection molding) over the magnetic assembly 1002, the one-or-more articulable joints 1212 can be optimized to achieve a desired balance between flexibility (e.g., bending radius) and magnetic field strength of the magnetic assembly 1002 in the distal portion of the stylet body 1000.

As to the primary layer 1206 being the reflowed layer, the reflowed layer can be molded around the core wire 104 or 138 and the magnetic assembly 1002 and subsequently reflowed around the core wire 104 or 138 and the magnetic assembly 1002 as shown in FIG. 20A. If present, one or more gaps between any two or more magnetic field-producing elements 106 can include polymeric material of the reflowed layer therebetween, thereby forming the one-or-more articulable joints 1212 in the magnetic assembly 1002. The polymeric material can be a thermoplastic polymer such as acrylic, acrylonitrile butadiene styrene (“ABS”), polyamide (e.g., nylon), polyether block amide (“PEBA”), polyurethane, polylactic acid, polybenzimidazole, polycarbonate, polyether sulfone, polyoxymethylene, polyether ether ketone (“PEEK”), polyetherimide, polyethylene, fluorinated ethylene propylene (“FEP”) polyphenylene oxide, polyphenylene sulfide, polypropylene, polystyrene, polyvinyl chloride, polyvinylidene fluoride (“PVDF”), or polytetrafluoroethylene (“PTFE”). Because the one-or-more gaps between the two-or-more magnetic field-producing elements 106 can be adjusted with respect to their widths before molding (e.g., injection molding) and reflowing the thermoplastic polymer over the magnetic assembly 1002, the one-or-more articulable joints 1212 can be optimized to achieve a desired balance between flexibility (e.g., bending radius) and magnetic field strength of the magnetic assembly 1002 in the distal portion of the stylet body 1000.

As to the primary layer 1206 being the potting layer, the potting layer can be potted around the core wire 104 or 138 and the magnetic assembly 1002 as shown in FIG. 20A. If present, one or more gaps between any two or more magnetic field-producing elements 106 can include potting material of the potting layer therebetween, thereby forming the one-or-more articulable joints 1212 in the magnetic assembly 1002. The potting material can be a thermosetting polymer such as epoxy, polyurethane, or silicone. Because the one-or-more gaps between the two-or-more magnetic field-producing elements 106 can be adjusted with respect to their widths before potting the potting material over the magnetic assembly 1002, the one-or-more articulable joints 1212 can be optimized to achieve a desired balance between flexibility (e.g., bending radius) and magnetic field strength of the magnetic assembly 1002 in the distal portion of the stylet body 1000.

As to the primary layer 1206 being the shrink-wrapped layer, the shrink-wrapped layer can be shrunk around the core wire 104 or 138 and the magnetic assembly 1002 as shown in FIG. 21A. If present, one or more gaps between any two or more magnetic field-producing elements 106 can include air or a spacer therebetween, optionally with some conforming shrink-wrapped layer shrunk into an outer diameter of the one-or-more gaps, thereby forming the one-or-more articulable joints 1212 in the magnetic assembly 1002. The shrink-wrapped layer can be a thermoplastic polymer such as a polyolefin (e.g., polyethylene, polypropylene, polybutene), a fluoropolymer (e.g., PTFE), or polyvinyl chloride (“PVC”). Because the one-or-more gaps between the two-or-more magnetic field-producing elements 106 can be adjusted with respect to their widths before shrinking the shrink-wrapped layer over the magnetic assembly 1002, the one-or-more articulable joints 1212 can be optimized to achieve a desired balance between flexibility (e.g., bending radius) and magnetic field strength of the magnetic assembly 1002 in the distal portion of the stylet body 1000.

Again, the outer construction 108 can be of a single layer (i.e., a single-layered outer construction) as shown in FIGS. 20A and 21A or multiple layers (i.e., a multi-layered outer construction) as shown in FIGS. 20B, 20C, and 21B. As to the multi-layered outer construction 108, such an outer construction can include the primary layer 1206 together with one or more other layers such as the secondary layer 1208, the tertiary layer 1210, etc. thereover or thereunder. For example, FIGS. 20B and 21B show the primary layer 1206 (e.g., the overmolded layer, the reflowed layer, the potting layer, or the shrink-wrapped layer) together with the secondary layer 1208 thereover. In such embodiments, the secondary layer 1208 can be a casing or tubing over the primary layer 1206. In another example, FIG. 20C shows the primary layer 1206 together with the secondary layer 1208 and the tertiary layer 1210 thereover. In such embodiments, the secondary layer 1208 can be a casing or tubing over the tertiary layer 1210, which can be a braided layer. When the primary layer 1206 is the reflowed layer, the polymeric material of the reflowed layer can be reflowed into the one-or-more gaps between any two or more magnetic field-producing elements 106 as well as into the braided layer, thereby forming a composite outer construction 108.

Lastly, methods include a method of using a magnetically trackable stylet. For example, such a method can include a catheter-inserting step, a catheter-advancing step, and a catheter-placing step. The catheter-inserting step includes inserting the catheter 72 into the insertion site 73 of the patient 70. The catheter 72 includes the stylet 100 or 130 disposed in a lumen of the catheter 72 such that the distal end 100B or 130B of the stylet 100 or 130 is substantially co-terminal with the distal tip 76A of the catheter 72. The catheter-advancing step includes advancing the catheter 72 through the vasculature of the patient 70 without breaking the stylet body 1000 of the stylet 100 or 130 due to bending-related fatigue. As set forth above, the stylet 100 or 130 includes the outer construction 108 around the magnetic assembly 1002 of the one-or-more magnetic field-producing elements 106 disposed in a magnetically trackable distal portion of the stylet body 1000 alongside the core wire 104 or 138. The catheter-placing step includes placing the distal tip 76A of the catheter 72 in a desired general region near the patient's heart in accordance with magnetic tracking of the TLS 50 of the integrated system 10 for placing the catheter 72.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures can be made from the particular embodiments disclosed herein without departing from the scope of the concepts provided herein.

1. A magnetically trackable stylet, comprising:
a stylet body having a magnetically trackable distal portion, the stylet body including:
a core wire;
a flexible magnetic assembly including one or more magnetic field-producing elements disposed alongside the core wire in the distal portion of the stylet body; and
a casing around the core wire and the magnetic assembly, the stylet body configured to be disposed in a lumen of a catheter for magnetically tracking a catheter tip in vivo without breakage of the stylet body due to bending-related fatigue.

2. The stylet of claim 1, wherein the core wire is tapered in the distal portion of the stylet body alongside the one-or-more magnetic field-producing elements.

3. The stylet of claim 1, further comprising a sealed stylet tip, the stylet tip including a seal sealing the one-or-more magnetic field-producing elements in the distal portion of the stylet body.

4. The stylet of claim 1, wherein the one-or-more magnetic field-producing elements include one or more polymer-bonded magnets, the one-or-more polymer-bonded magnets configured to bend and, thereby, allow the stylet to bend in accordance with an anatomy of a patient without breaking.

5. The stylet of claim 4, wherein the one-or-more polymer-bonded magnets include a single cylindrical polymer-bonded magnet.

6. The stylet of claim 4, wherein the one-or-more polymer-bonded magnets include a plurality of cylindrical polymer-bonded magnets.

7. The stylet of claim 1, wherein the one-or-more magnetic field-producing elements include one-or-more sintered magnets.

8. The stylet of claim 7, wherein the one-or-more sintered magnets include a plurality of cylindrical sintered magnets having radiused ends, the radiused ends of the cylindrical magnets configured to allow the cylindrical magnets to bend into each other and, thereby, allow the stylet to bend in accordance with an anatomy of a patient without breaking.

9. The stylet of claim 7, wherein the one-or-more sintered magnets include a plurality of cylindrical sintered magnets alternating with a plurality of spherical sintered magnets forming articulable joints therebetween, the joints configured to allow the cylindrical magnets to bend into each other and, thereby, allow the stylet to bend in accordance with an anatomy of a patient without breaking.

10. The stylet of claim 7, wherein the one-or-more sintered magnets include a plurality of cylindrical sintered magnets alternating with a plurality of non-metallic spheres forming articulable joints therebetween, the joints configured to allow the cylindrical magnets to bend into each other and, thereby, allow the stylet to bend in accordance with an anatomy of a patient without breaking.

11. The stylet of claim 7, wherein the one-or-more sintered magnets include a plurality of cylindrical sintered magnets alternating with a plurality of septa sectioning a groove of a magnet holder in which the sintered magnets are disposed, the septa forming articulable joints between the cylindrical magnets configured to allow the cylindrical magnets to bend into each other and, thereby, allow the stylet to bend in accordance with an anatomy of a patient without breaking.

12. The stylet of claim 7, wherein the one-or-more sintered magnets include a plurality of cylindrical sintered magnets disposed in a plurality of magnet holders forming articulable joints therebetween, the joints configured to allow the cylindrical magnets to bend into each other and, thereby, allow the stylet to bend in accordance with an anatomy of a patient without breaking.

13. The stylet of claim 12, wherein each magnet holder of the magnet holders includes a ball end and a socket end, the joints being ball-and-socket joints.

14. The stylet of claim 12, wherein the magnet holders include ball-ended magnet holders having a pair of ball ends and socket-ended magnet holders having a pair of socket ends, the joints being ball-and-socket joints.

15. The stylet of claim 12, wherein the magnet holders are links and the joints are interlinks between the links chained together.

16. The stylet of claim 1, wherein the one-or-more magnetic field-producing elements include one or more magnetic wires twisted or braided with the core wire, the one-or-more magnetic wires configured to bend and, thereby, allow the stylet to bend in accordance with an anatomy of a patient without breaking.

17. A magnetically trackable stylet, comprising:
a stylet body having a magnetically trackable distal portion, the stylet body including:
a flexible magnetic assembly of one or more magnetic wires in the distal portion of the stylet body; and
a casing around the magnetic assembly, the stylet body configured to be disposed in a lumen of a catheter for magnetically tracking a catheter tip in vivo without breakage of the stylet body due to bending-related fatigue.

18. The stylet of claim 17, wherein the one-or-more magnetic wires include a single magnetic wire.

19. The stylet of claim 18, further comprising a core wire in the distal portion of the stylet body, the magnetic wire twisted with the core wire.

20. The stylet of claim 18, further comprising a core wire in the distal portion of the stylet body, the magnetic wire helically wrapped around the core wire.

21. The stylet of claim 17, wherein the one-or-more magnetic wires include a plurality of magnetic wires.

22. The stylet of claim 21, further comprising a core wire in the distal portion of the stylet body, the magnetic wires twisted or braided with the core wire.

23. The stylet of claim 21, further comprising: a core wire in the distal portion of the stylet body, the magnetic wires twisted or braided around the core wire.

24. The stylet of claim 17, further comprising a sealed stylet tip including a seal sealing the magnetic wires in the distal portion of the stylet body.

25-49. (canceled)
